(19) United States
(12) Patent Application Publication
(10) Pub. No.: US 2015/0343142 A1
(43) Pub. Date: Dec. 3, 2015

(54) DEVICE

(71) Applicant: MEDICEN DEVISE LIMITED, Edinburgh (GB)

(72) Inventor: Kanika BANSAL, Edinburgh (GB)

(21) Appl. No.: 14/653,527

(22) PCT Filed: Dec. 17, 2013
(86) PCT No.: PCT/GB2013/053327

§ 371 Date: Jun. 18, 2015

(30) Foreign Application Priority Data
Dec. 18, 2012 (GB) 1222782.3

(51) Int. Cl. A61M 5/172; A61M 35/00

(52) U.S. Cl. CPC A61M 5/172; A61M 35/00

(57) ABSTRACT

Surgical puncture/incision wounds and/or medical/surgical equipment are vulnerable to contamination and/or infection and require labour intensive maintenance by regularly administering antibiotics and/or checking, cleaning and replacing dressings. The present invention obviates the problems associated with the prior art by providing an automated device that can be programmed to deliver to a wound and/or a piece of medical/surgical equipment, an agent to prevent or treat infections.

### FIELD OF THE INVENTION

The present invention provides devices which may be used to treat or prevent wound infections

### BACKGROUND OF THE INVENTION

The skin forms part of the innate immune system of humans and animals, it represents the first barrier against infection. Any breach of the skin is vulnerable to infection and, without proper dressing or treatment, may become contaminated with microorganisms. While wounds can occur as a consequence of an accident or trauma, surgical procedures often leave wounds which require careful maintenance in order that they heal and/or remain free of infection.

Particularly problematic are surgical puncture or incision wounds which may be present for extended periods of time. For example, IV drips exploit needles inserted through the skin and into vessels—the needles may be left in situ for prolonged period of time. As a consequence, the resulting puncture wound is vulnerable to infection, not only from skin commensal microorganisms but from other pathogens present in the community.

Wound maintenance is labour intensive and nurses must regularly administer antibiotics and/or check, clean and/or replace dressings.

U.S. Pat. No. 5,620,424 (A) describes a device impregnated with or containing a supply of antiseptic agent, which device may be attached to a catheter to prevent infections at the catheter site. The device is passive and cannot be programmed to regularly dispense antiseptic agent, therefore it may perpetuate the problem of drug resistance in microorganisms. Furthermore, the location of the device on the surface of the treated skin obscures the visualisation of the wound.

U.S. Pat. No. 4,834,711 (A) describes an appliance for use in insertion of a catheter (especially aimed for use with urinary-catheters); the appliance comprises a transparent shield to cover the area under treatment and a guide conduit through which a catheter may be thread. The device may be used to apply a disinfectant, lubricant or analgesic substance to a catheter as it is passed through the appliance.

US2010256607 (A1) describes a sterilisation system for continuously or intermittently sterilising implanted prosthetics or catheters (externally and/or internally) by means of ultrasounds, radiation or a pumped drug.

U.S. Pat. No. 5,338,308 (A) describes an assembly for prevention of catheter sepsis where the catheter is inserted through a skin surface. The assembly comprises a patch covering the area of the skin surface under treatment and an ancillary tube disposed between the patch and the skin with an inlet through which a needle can be inserted to apply an antiseptic agent. This assembly does not permit wound visualisation while in use wound visualisation. Moreover, the assembly cannot be releasably attached to different pieces of medical/surgical equipment not can the position of the assembly relative to the wound and/or catheter be changed.

U.S. Pat. No. 3,981,299 describes a urethral catheter device having a tubular extension designed to permit the injection of antibiotics or anaesthetic onto the outer wall of the catheter. The present invention provides an auxiliary device which can be attached to any type of medical device including, for example, catheters and manipulated such that it delivers medicated fluids to both the puncture wound and/or the device.

U.S. Pat. No. 6,432,100 describes a protective sleeve formed of a biocompatible polymer which protects percutaneous leads from infection.

US2002078963 provides a cartridge for use on respiratory, lavage and gut/respiratory monitoring surgical equipment. Specifically, the document discloses that the cartridge may comprise an antimicrobial hydrogel which “wipes” the surgical instrument as it is inserted and retracted through the cartridge. The cartridge system described by US2002078963 is a passive system requiring movement of the surgical instrument to achieve the necessary “cleaning”. There is no disclosure of an automated and programmable fluid delivery mechanism to ensure constant and complete surface decontamination.

US2003167050 describes a sponge like material comprising an anti-microbial fluid which can be places around a catheter to form an anti-microbial barrier. Since the anti-microbial fluid is consumed over time, the sponge has a finite shelf life and there is no disclosure of any system to continually apply fresh anti-microbial fluid to the sponge.

Moreover, the sponge may occlude the wound site impairing wound visualisation and promoting further infection—particularly once the anti-microbial fluid has expired or dried and if the sponge is not promptly moved.

US2008172011 provides catheters comprising water-soluble, antimicrobial agents in an outer layer of a sleeve. This is a passive system comprising a consumable antibiotic which is not automatically replaced.

While the prior art has sought to provide solutions to these problems, many of the approaches are inconvenient due to complexity and occlusion of the wound site, encouraging further infection and preventing the medical staff from being able to follow the healing and/or development of the wound without having to remove, move or displace the equipment.

The present invention seeks to obviate one or more of the problems associated with the prior art.

### SUMMARY OF THE INVENTION

In a first aspect, the present invention provides an automated device for applying, delivering, discharging or dispensing an agent to a biological tissue and/or a wound. The device may be configured such that, in use, the device does not occlude, obscure or cover the wound. Moreover, in use, the device may permit wound visualisation such that the healing, development and/or infection status of the wound may be monitored without need to move, remove or displace the device.

In use, the automated device of the first aspect of this invention may be used to ensure the regular application, delivery, discharge or dispensing of an agent to a biological tissue and/or a wound.

The agent may comprise a powder or a fluid. Thus the agent may be described as a fluid (a fluid agent)

The agent may comprise one or more components for preventing, suppressing or treating an infection, disease, condition or syndrome. The agent may comprise one or more components for decontaminating, cleaning or disinfecting a biological tissue or any type of surface. For example, the agent may comprise one or more of an antibiotic, an antiseptic, a disinfectant and/or a drug.

Where the agent is a fluid, the fluid may be a medicated fluid. A medicated fluid may comprise one or components for preventing, suppressing or treating an infection, disease, condition or syndrome.

As such, the present invention provides an automated device for applying a fluid or medicated fluid to a wound and/or a biological tissue, said device comprising a reservoir for storing said fluid or medicated fluid and means for automatically applying a volume of the fluid or medicated fluid to a wound and/or a biological tissue.

It should be understood that hereinafter, all references to a “fluid” encompass medicated fluids of the type described above.

The agent or fluid may further comprise a pharmaceutically acceptable diluent, excipient or carrier.

The agent or fluid may comprise an antiseptic.

The agent or fluid may comprise an antibiotic. For example, the agent or fluid may comprise an anti-bacterial, an anti-viral and/or an anti-fungal agent.

The agent or fluid may comprise a disinfectant.

The agent or fluid may comprise one or more of an antiseptic, an antibiotic and/or a disinfectant.

Any of the agents or fluids described herein may further comprise one or more additional components such as one or more drugs or other therapeutic agents. For example, the agent or fluid may further comprise a chemotherapeutic agent, an antiinflammatory agent, an aesthetic or analgesic agent.

A part (component) or parts of the automated device may be disposable or replaceable. Alternatively, the automated device may be disposable.

The present invention may provide an automated device for applying a fluid to a wound and/or a biological tissue, said device comprising a reservoir for storing said fluid and means for automatically applying a volume of the fluid to a wound, wherein the fluid comprises an antiseptic.

The term “wound” encompasses any type of flesh wound, such as, for example a skin or tissue wound. A skin or tissue wound may constitute a break in the skin or tissue. The wound may be an open wound or may be dressed. The wound may be present on a subject in a hospital or other type of care centre (for example a residential care home, local surgery or the like). The wound may have occurred as a consequence of an accident or may be a surgical wound. The term “surgical” wound encompasses any type of wound created by a surgical procedure and may include, for example puncture wounds, incision wounds or the like. A surgical wound may occur at a “surgical site”. The wound may be a puncture wound caused by or associated with, the insertion of a surgical device or object such as, for example a needle, tube, cannula or catheter into and through the skin or tissue of a subject. Wounds of the type described herein may comprise a surgical device or object (for example a needle, tube, cannula or catheter) in situ. A surgical wound may comprise an insertion site, a catheter insertion site or a catheterisation site.

The term biological tissue may encompass any internal or external human or animal tissue including, for example, the skin, mucosal tissues, epithelial tissues, surfaces of the gut and the like.

One of skill will appreciate that biological tissues and/or wounds—in particular skin and or tissue wounds, are vulnerable and/or susceptible to infection. For example, commensal skin microorganisms such as Staphylococcus and Candida species may contaminate and infect a tissue or a wound. Other microorganisms such as species belonging to the Escherichia, Pseudomonas and Streptococcus genera may also contaminate and infect tissues or wounds. Infection is a particular problem in surgery (bother major and minor surgery) where the skin barrier is breached through puncture or incision by needles, knives, tubes, cannula, catheters and the like. For example, the insertion of a needle into and through the skin may contaminate the wound with skin commensal organisms and other opportunistic pathogens which grow to form an infection. While the infection may be local and self-contained, it may spread to cause a septicaemia.

In addition to finding application in the treatment and/or prevention of biological tissue and/or wound infections, the devices of this invention may be further used to decontaminate, clean and/or disinfect medical devices and/or equipment. The term “medical device/equipment” may embrace, for example surgical devices/instruments and/or tools such as, for example needles, catheters and/or cannula—indeed the term should embrace any device used to deliver a composition intravenously. One of skill will further appreciate that medical equipment—in particular catheters and the like, is susceptible to the formation of biofilms and contamination by microorganisms. Thus medical equipment also poses an infection risk especially when that equipment passes through the skin of a patient. For example, catheters, may, in use, become contaminated by commensal microorganisms; those commensal organisms may then grow on the surface of the catheter, occasionally forming a biofilm. In the most serious cases, microorganism on the surface of catheters can get into the blood stream of the patient increasing the risk of septicaemia. For convenience, the term “catheter” may be used in place of the terms “medical device” or “medical equipment”.

Given that wounds and/or medical equipment are susceptible to infection, the devices described herein may find application in the treatment and/or prevention of equipment contamination and/or wound and/or surgical site infections. Without wishing to be bound by theory, it is suggested that the devices of this invention may be exploited to affect the regular application of a medicated (for example antiseptic) agent or fluid to medical equipment/devices and/or wounds so as to prevent or treat the occurrence of contamination or an infection.

The devices provided by this invention are automated. That is to say they are capable of delivering or dispensing a quantity of agent or fluid to a wound or equipment without human input. The devices of this invention may be programmed or configured so as to dispense or deliver a predetermined quantity of agent at a predetermined time or times. For example, the devices of this invention may deliver a quantity of agent to a wound or equipment at regular, predetermined times and/or intervals.

Additionally, or alternatively, the devices provided by this invention may also be activated to deliver a quantity of agent to a wound or equipment. Such activation may be via user (for example human) interaction with the device. For example, in addition to the device being programmed or configured to automatically deliver or dispense an agent, a user (perhaps a patient) may activate the device to deliver or dispense a quantity of agent. The devices of this invention may be remotely controlled.

The devices of this invention may include one or more environmental sensors such that the delivery of an agent to a wound or equipment is activated in response to one or more environmental stimuli. For example, the device may comprise motion, temperature (patient and/or environmental temperature), moisture (humidity), pressure, light and/or sound sensors. One of skill will appreciate that any given sensor may be configured to activate the device to dispense or release a quantity of agent in response to a suitable stimuli threshold.

The agent to be delivered, applied, dispensed and/or discharged by the devices described herein may be sprayed, squirted, misted or dropped on to a wound, surface or equipment.

The device may comprise, or further comprise, a body or housing.

The body or housing may be a single part or may be multi component.

The housing may define an aperture through which the agent is dispensed or discharged.

The devices of this invention may comprise a reservoir for storing agent. The reservoir may take the form of a tank, vessel, canister or receptacle. The reservoir may be a single part or may be multi component. The reservoir may be air and/or water tight. The reservoir may be a sealed and pre-filled with an agent. Alternatively, the reservoir may be user filled and re-filled. In such cases, the reservoir may comprise a releasable seal or cap covering a port through which agent may be delivered to the reservoir.

A device of this invention may comprise a reservoir and a housing/body part which are connected together and in fluid communication with one another. For example, the reservoir may comprise a fluid outlet port through which agent stored in the reservoir may flow or pass into the body/housing portion of the device. The body or housing portion may further comprise a fluid inlet port adapted to receive the fluid outlet port of the reservoir. The fluid outlet/inlet ports of the reservoir and housing/body portions may define fluid conduits which communicate fluid between the reservoir and the housing/body portion. The housing/body portion of the device may comprise a further fluid outlet port via which fluid may exit the housing/body portion.

The devices of this invention may comprise a plurality of (i.e. two or more) reservoirs.

The body or housing of the device may be configured to receive, for example releasably receive, a reservoir. Accordingly, the reservoir may be releasably contained within the device. The reservoir may be adapted to be locked into the body or housing of the device. For example the reservoir may comprise one or more “key”-like projections which can be inserted into an aperture defined by the housing. Once inserted, the reservoir may be orientated to move the “key”-like projection, from a first position to a second position which locks the reservoir to the body/housing of the device.

The reservoir and device may be integrally formed.

The reservoir may be remote from the device and connected thereto by means of a conduit or tube through which the agent (for example fluid) stored or held in the reservoir, may be transmitted.

The reservoir may comprise or connect to a spray head, (fluid or agent) applicator or nozzle through which an agent, for example a fluid, or medicated fluid (stored in the reservoir), may be dispensed or discharged. The body or housing of the device may define an aperture or outlet through which a spray head, applicator or nozzle may project or dispense/discharge a quantity of agent to a wound.

The devices of this invention may comprise one or more nozzles, applicators or spray heads through which agent is dispensed, delivered or discharged. The nozzles, applicators or spray heads may be connected directly (or indirectly) to the reservoir. The nozzles, applicators or spray heads may be attachable or releasably attachable to (medical) equipment, surfaces or body parts. The nozzles, applicators or spray heads may be part of a tube and/or connected to a tube.

The tube may be a flexible tube. The tube may be attached directly (or indirectly) to the device of this invention and/or to any reservoir or body/housing portion thereof. The devices of this invention may comprise one or a plurality of flexible tubes. The tubes may be straight with a bendable portion. The tubes may be branched having multiple spray heads, applicators and/or nozzles through which fluid may be discharged, sprayed, dispensed or delivered.

The spray head, applicator or nozzle of the device may be remote or distal from the reservoir and/or body part(s) of the device and connected thereto by some form of fluid conduit, for example a flexible tube. This allows the body/reservoir portion of the device to be attached at one location and the spray head. Applicator or nozzle at another location.

The spray head, applicator or nozzle may be formed and adapted to spray, drop or otherwise deliver an agent, for example a fluid agent, to, for example a surface, wound or a biological tissue.

The device of this invention and/or reservoir thereof may comprise means for forcing fluid from the reservoir to and out of any spray head, applicator or nozzle. For example, the device may comprise a pressurised canister or other vessel. The pressurised canister may hold agent to be delivered to a wound. The pressurised canister may contain a propellant. The pressurised canister may further comprise a valve. The valve may be a one-way valve biased to the closed position. The one-way valve may be configured to allow pressurised agent to pass therethrough upon opening.

The device may further comprise means to affect the opening of the canister (or valve thereof) so as to dispense, deliver or discharge a quantity of agent therefrom. The means for opening the valve may comprise and motor which drives or operates one or more actuator members. The actuator members may engage with the canister to open the valve thereof and affect the discharge or dispensing of a quantity of agent.

The reservoir (or reservoirs) may be connected to one or more other canisters or vessels. The reservoirs may be connected to the one or more other vessels or canisters by one or more tubes which define flow conduits. In use, agent held within the reservoir may flow to these other canisters or vessels through the conduits defined by the connecting tubes. Agent may be pumped from the reservoir, through a connecting tube into a vessel or canister. Additionally, or alternatively, agent may pass between the reservoir and vessel/canister (via connecting tube) under the action of gravity.

The reservoir and/or vessel/canister connected thereto may comprise a piston. The piston may be moved to apply a pressure to the agent within the reservoir such that a quantity of agent is discharged or dispensed therefrom. The piston may be operated by one or more motors.

The reservoir and/or vessel/canister may comprise a pump. In use, the pump may be adapted to pump agent (for example fluid or medicated fluid) from the reservoir to a vessel or canister or from the vessel/canister and on to a wound.

The devices described herein may co-operate with other types of equipment, surfaces or with body parts. In particular, the devices of this invention may co-operate with medical equipment, surfaces and/or patient body parts. For example, the devices may be formed and adapted so as to be attachable, for example releasably attachable, to other types of equipment, surfaces or body parts.

The devices of this invention may be comprise a connector for gripping or attaching (for example releasably attaching) to, other equipment, a surface or a body part.

The device may comprise a clip.

The device may comprise a magnet or magnetic component for attaching to a magnet or magnetic surface.

The device may comprise a threaded connector for attaching to a corresponding connector on another device.

The device may comprise a strap

The device may comprise a hook and loop connector system—for example Velcro®.

The device may comprise an interference fit element which releasable connects with medical equipment, in particular, for example, intravenous lines, catheters, tubes, needles and the like.

The devices described herein may be releasably attachable or attachable to an intravenous line. The devices may be releasably attachable to a body part.

The devices may comprise a strap or clip to enable the device to be attached or releasably attached to another device or a body part.

The different components of the device may be releasably attached or clipped to existing medical equipment at different heights and locations.

A device of this invention may comprise a body section comprising a reservoir for storing an agent to be delivered to a wound or some piece of equipment (for example medical equipment) and a flexible tube connecting the body section of the device to a fluid applicator or nozzle. The connecting piece of flexible tube may define a conduit through which fluid (for example agent as described herein) may pass. In this way, the flexible tube serves to communicate fluid from the reservoir to a remote nozzle or applicator, which nozzle or applicator is configured/adapted to dispense, discharge or deliver agent to, for example, a piece of equipment, a biological tissue and/or a wound. The body section may comprise a clip or grip element as described herein permitting it to be connected a piece of medical equipment, for example an intravenous line or a catheter. The nozzle or applicator may be formed and adapted so as to permit it also to be releasably connected to a piece of medical equipment. The body part and the applicator nozzle may be connected to different parts of the same piece of medical equipment or to different pieces of medical equipment. For example, the body part (which may include a reservoir component) of the device of this invention may be attached or affixed to an intravenous line and the nozzle or applicator part (which dispenses, discharges or delivers agent to a piece of equipment and/or to the wound and which is connected to the body part by a length of flexible tube) attached or affixed (or somehow associated with) a catheter, needle or other piece of equipment/tool attached to the intravenous line. The catheter, needle or other tool may penetrate the skin of a patient and the applicator or nozzle may be located on the catheter, needle or tool at, near or adjacent the skin and/or the wound (puncture wound) caused by passage of the catheter/needle or tool through the skin. The applicator or nozzle of a device of this invention may be located adjacent to the wound but not in contact therewith—this may facilitate wound visualisation.

As stated, the nozzle or applicator of a device of this invention may be adapted deliver, dispense or discharge (by spraying, dropping, squirting or the like) a quantity of fluid onto a piece of equipment or a wound. Where the fluid is medicated fluid or an agent as described herein, for example an antiseptic or antibiotic agent) the regular application of the fluid (agent) on to a surface, piece of equipment (for example a catheter), biological tissue and/or a wound, may facilitate the suppression of an infection. The nozzle or applicator may be further adapted to dispense, discharge and or deliver a quantity of agent to substantially all or a major part or portion of a tubular piece of medical equipment—for example a catheter. The nozzle or applicator may take any form or shape but may define a space or gap into which a piece of equipment or a device (for example a medical device) may be located. For example, when in situ (i.e. penetrating the skin of a patient) an applicator or nozzle of this invention may be placed against or around a catheter.

The applicator or nozzle may comprise an annular member or ring structure. By its very nature, a nozzle or applicator which takes the form of an annulus or ring, defines a central aperture or bore through which a tubular piece of medical equipment, for example a catheter, may pass. The applicator or nozzle may comprise a partial annulus or ring structure.

An inner surface of the nozzle/applicator (perhaps an annulus), for example the inner surface which defines a central aperture or bore, may further define one or more fluid outlet apertures. Each fluid outlet aperture may define the opening to a fluid conduit or bore which pass through the structure or body of the applicator/nozzle to one or more fluid inlet ports through which agent to be delivered to a wound or piece of equipment (for example a catheter) may be passed.

The fluid conduit(s) which passes/pass through the annular or ring structure of the applicator or nozzle, may connect or extend to a single fluid inlet port. The fluid conduit(s) which passes/pass through the annular or ring structure of the applicator or nozzle, may connect or extend to a plurality of fluid inlet ports.

The fluid inlet port of the applicator nozzle may be connected to the reservoir of a device of this invention.

The inner surface of the nozzle/applicator (perhaps having an annular or ring structure), may define a recessed surface, which, in use forms or defines a channel through which a fluid (for example an agent as defined in this invention) may flow. The recessed surface may extend circumferentially around the inside surface of the applicator/nozzle. In use and when an applicator or nozzle of a device of this invention is placed around, for example, a tubular medical device (for example a catheter), the circumferentially orientated recessed surface and the outer surface of the tubular medical device may define a circular or curved channel through which fluid may flow. One of skill will appreciate that a channel of this type would allow fluid (for example a medicated fluid agent) to flow around all or part of the outside surface of the medical device. Where the fluid is a medicated agent, this would facilitate the cleaning, disinfection and/or decontamination of the medical device.

For example, the recessed surface may extend circumferentially around a lower edge of the inner surface of the (annular/ring) nozzle/applicator. One of skill will appreciate that fluid communicated (under pressure) from the reservoir of the device, may be excreted from the fluid outlet aperture defined by the inner surface of the applicator/nozzle structure. The fluid may then flow along the recessed surface. The fluid may be caused to flow along the recessed surface by capillary or wicking action.

The surface properties of the recessed surface (the “capillary surface”) may be modified so as to increase the surface wettability, for example by the provision of a wicking material, or by a surface treatment such as a coating (e.g. of an oxide or hydrophilic resin material) or a roughening surface treatment, for example by exposing the surface to a plasma, by laser ablation, etc. Other parts of the applicator/nozzle of the device of this invention may comprise one or more fluid stop features, which are designed to prevent a fluid from passing through the stop feature, by virtue of capillary action alone. For example, surfaces of the applicator/nozzle, other than the recessed surface, may be provided with hydrophobic material(s) (e.g. printable conductive or non conductive inks) or a process or material that changes surface properties therefore creating a hydrophilic/hydrophobic differential (e.g. by way of laser ablation, surface scoring, surface material removal, evaporated metallic materials etc), which is designed to about/be a wall feature or is coated on a wall of the channel.

In use, the recessed surface of the inner surface of the applicator/nozzle may permit fluid to flow around all or substantially all, of the outer surface of any catheter type device passing through the applicator/nozzle into and through the skin. In use, and without wishing to be bound by theory, there may form, between the recessed surface and any, for example, catheter passing through applicator/nozzle, a capillary gap which facilitates the flow of fluid from the fluid outlet aperture around the outside surface of the catheter. In this way, all, substantially all or at least a part of the outside surface of the catheter may be kept clean and free of microbial contamination. The recessed surface may further facilitate the distribution of any agent excreted from the fluid outlet aperture of the inner surface of the applicator to a wound.

The recessed surface of the applicator/nozzle ensures that even the smallest quantity of fluid sprayed, dropped or delivered from the fluid outlet aperture defined by an inner surface of the applicator/nozzle, is maximally distributed around the inner surface of the applicator nozzle and across an outer surface of any device (for example catheter) passing through the applicator/nozzle.

The applicator or nozzle may comprise a clear material or one or more “window” type structure(s) such that any wound below the applicator/nozzle is visible.

Thus, the invention may provide a device for delivering an agent to a wound and/or a piece of medical equipment, for example a catheter type device, the device comprising a reservoir which is releasably connectable to an intravenous line and an agent applicator which can be releasably attached to any equipment or a catheter type device extending from the intravenous line, wherein agent stored in the reservoir is communicated to the applicator by a conduit and further wherein the applicator defines a fluid outlet aperture through which fluid may pass to the surface of the medical equipment/catheter and/or wound. The applicator may comprise a surface which facilitates the transmission or flow of fluid passing through the fluid outlet aperture around an inside surface of the applicator and/or the outside surface of the medical equipment (for example catheter). The surface may be recessed such that optionally in co-operation with a surface of the medical device (for example catheter), it defines a channel or recess through which fluid may flow. In use, the surface or recessed surface (again optionally in co-operation with a surface of the medical device) may form a capillary gap through which fluid may flow.

Any of the devices described herein may further comprise one or more device status/performance indicators. A device status/performance indicator may serve to inform or warn users of, for example, malfunction, low or loss of power, low battery life, low/depleting/expired agent levels and the like. A status/performance indicator may take the form of one or more audible and/or visual indicators and may include, for example alarms and/or lights (for example LED type indicators). Any status or performance indicator may sound or light up or change colour to alert a user as to some issue with the performance and/or status of the device.

An advantage of the present invention over prior art devices is the targeted delivery of agent to specific parts of medical/surgical equipment (for example a catheter) or a wound. This is in part brought about by the applicator feature which insofar as it is moveable and connected to a reservoir of agent by a flexible conduit, can be positioned accurately at a defined or predetermined location on a piece of medical equipment and/or at or near a wound to ensure the targeted delivery, dispensing and/or discharging of agent. Moreover, the recessed surface of the applicator which (in use) defines a fluid (or agent) flow channel (or capillary gap or capillary/wicking path), ensures that agent dispensed, delivered and/or discharged from the applicator is dispersed and/or spread around an outside surface of the medical device or equipment. In use (and for example) where the medical/surgical equipment comprises a tubular device (for example a catheter), the recessed surface of the applicator may run parallel to and in close proximity with, an outside surface of the medical/surgical equipment such that fluid or agent flowing through the channel formed by the recessed surface, is in contact with and flowing across or along at least part of the outside surface of the medical/surgical equipment. In this way all or part of an outside surface of a piece of medical equipment may be prevented from becoming contaminated and/or dirty. At this point, and under gravity agent may flow down the medical equipment (for example the catheter) towards an associated wound.

The present invention provides a device or devices as substantially described in the description and figures of this specification.

Being attachable to other types of equipment, for example intravenous lines and the like, allows the devices of this invention to be located at or near the site of a surgical (for example puncture) wound without obscuring it in order to provide wound visualisation. The device may then be used to regularly and controllably deploy, dispense, discharge or apply a quantity of an agent (for example an antiseptic) to the wound so as to prevent or minimise the risk of a wound infection developing.

The device provided by this invention may have a range of other uses. For example, the device may be exploited in order to apply, deliver, dispense or discharge an agent to surfaces, contact surfaces, air, materials, equipment (for example surgical equipment) and the like. For example, the device may be exploited as a means to (automatically and/or regularly) deliver an agent to surfaces regularly in contact with humans, animals and/or food—for example beds, clinical and/or surgical equipment. gym equipment, doors, door handles, widows, ceilings, floors, walls, furniture, vehicles (both interior and exterior parts), tables, food, food preparatory surfaces and the like. As described in more detail below, the agent may comprise one or more components for preventing, suppressing or treating an infection, disease, condition or syndrome. The agents may comprise one or more of an antibiotic, an antiseptic, a disinfectant and/or a drug.

Thus in a further aspect, the invention provides a (or an automated) device for applying, delivering, discharging or dispensing an agent to the air, materials, tools, and/or a surface/contact surface. For the avoidance of doubt the definitions used throughout this specification apply to the corresponding terms and features of this final aspect of the invention.

### DETAILED DESCRIPTION

The present invention will now be described in detail with reference to the following Figures which show:

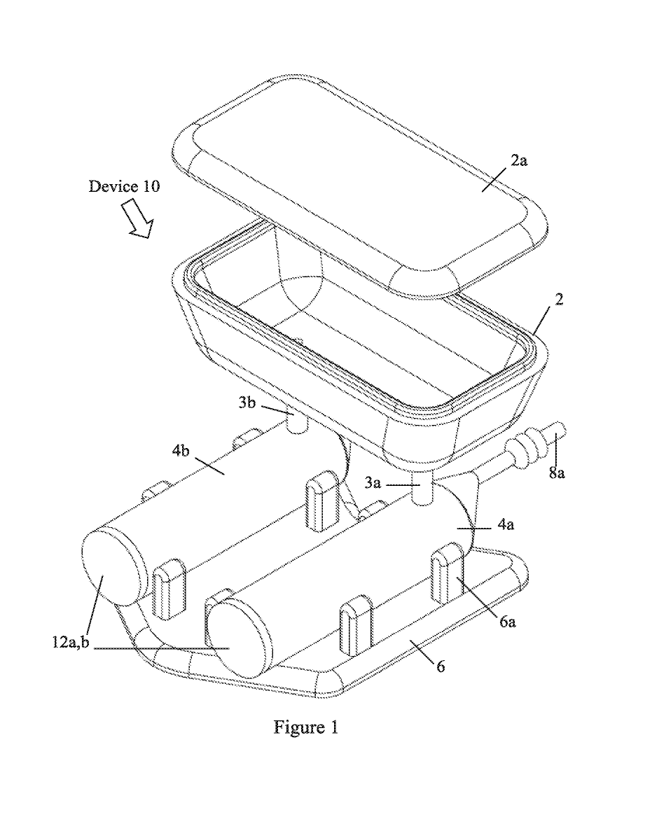

FIG. 1: Is an exploded perspective view of a device according to one embodiment of this invention

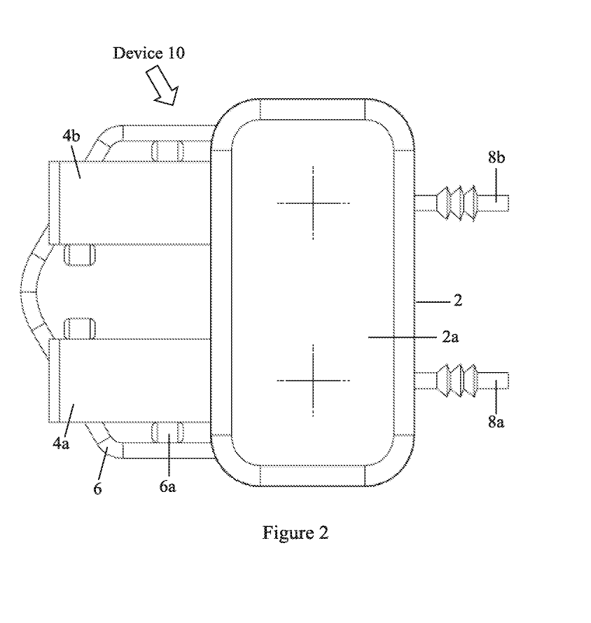

FIG. 2: is a plan view of the top surface of the device shown in FIG. 1

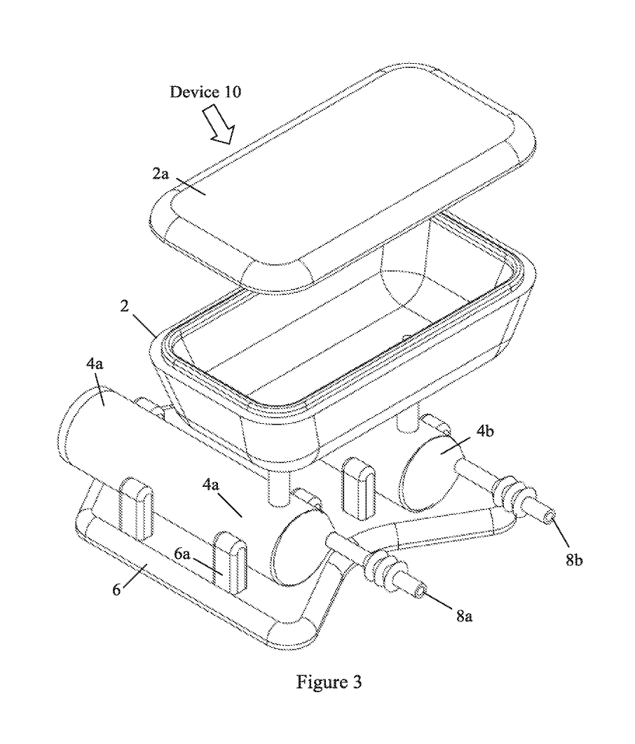

FIG. 3: is another exploded perspective view of a device according to an embodiment of this invention.

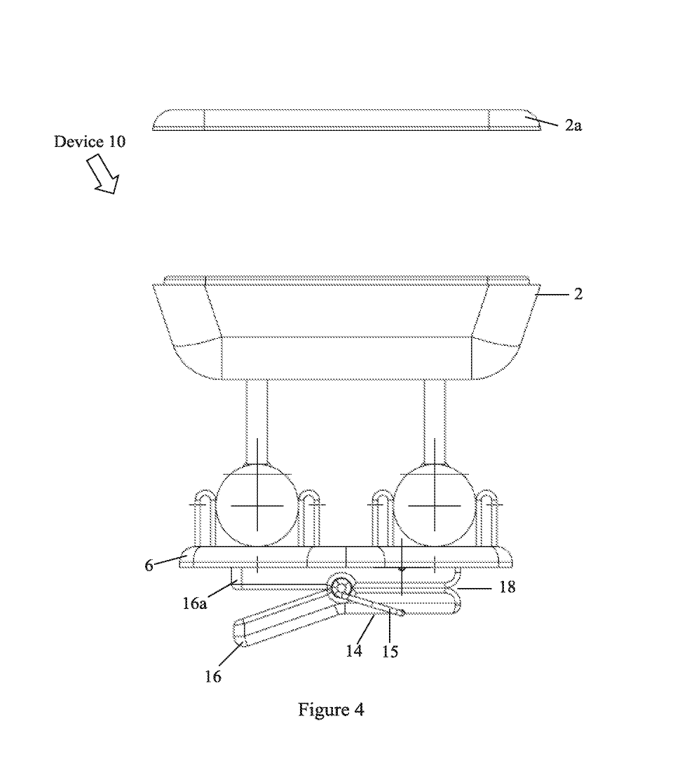

FIG. 4: is a plan view of an end of the device shown in FIGS. 1 and 3.

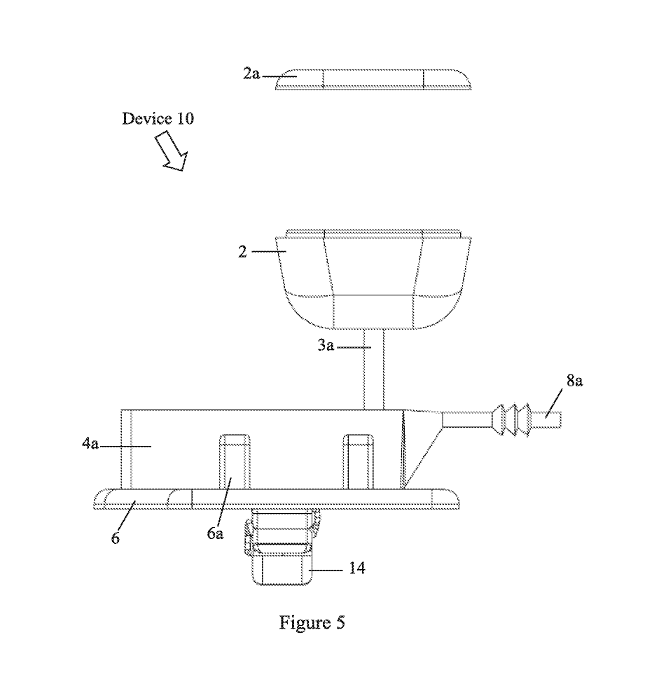

FIG. 5 is a plan view of the side of the device shown in FIGS. 1 and 3.

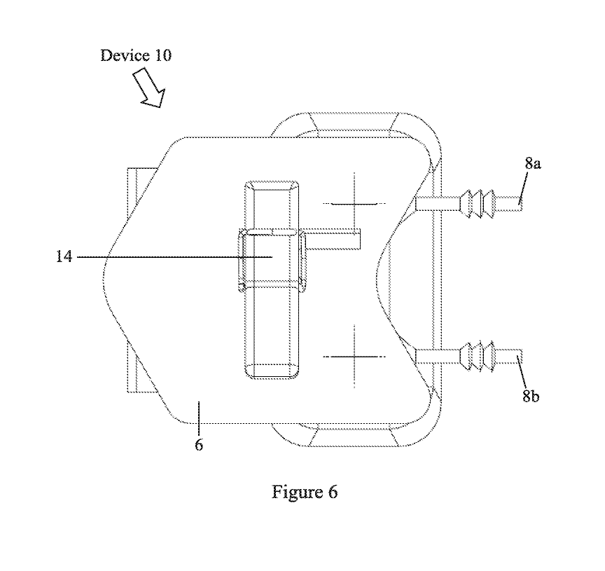

FIG. 6 is a plan view of the bottom surface of the device shown in FIGS. 1 and 3.

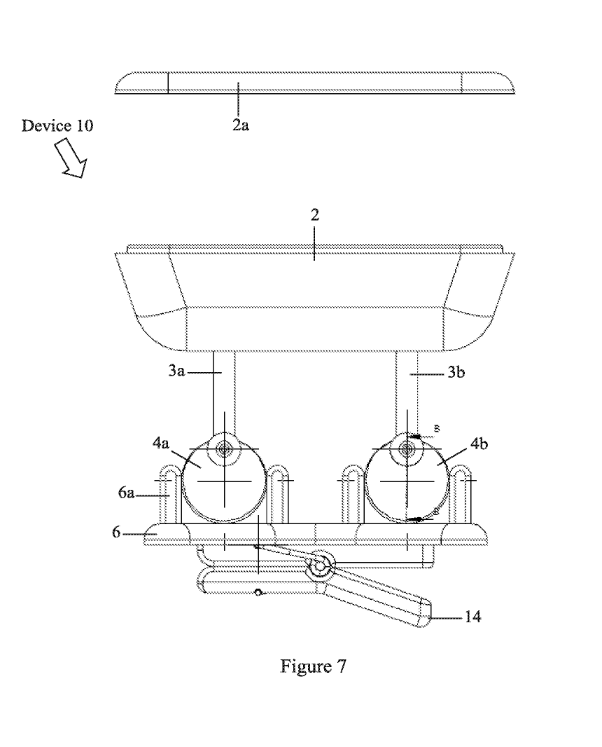

FIG. 7: is a plan view of the other end of the device

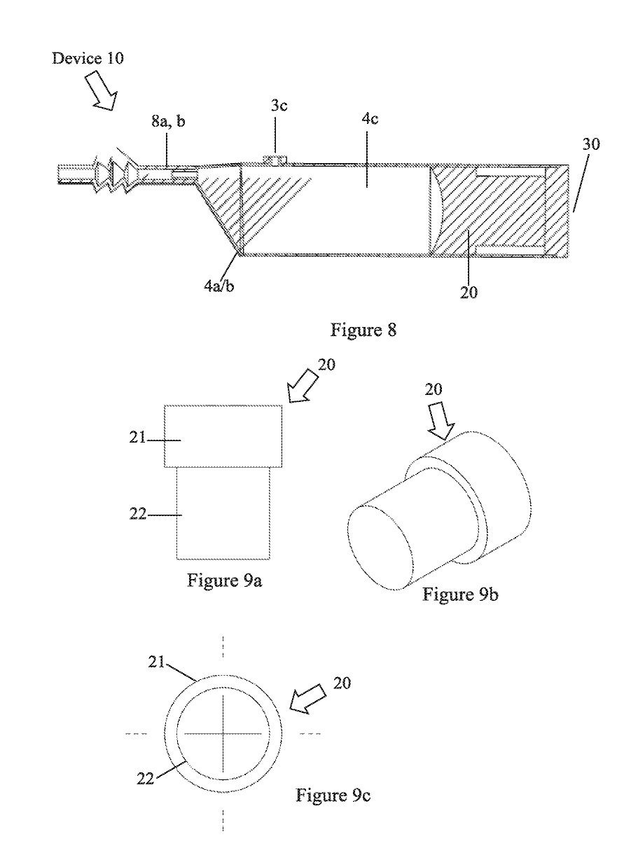

FIG. 8: shows a plan view of the side of a component of the device shown in FIGS. 1 and 3.

FIGS. 9a-c: plan, perspective and end views of a piston component used in the device shown in FIGS. 1-8.

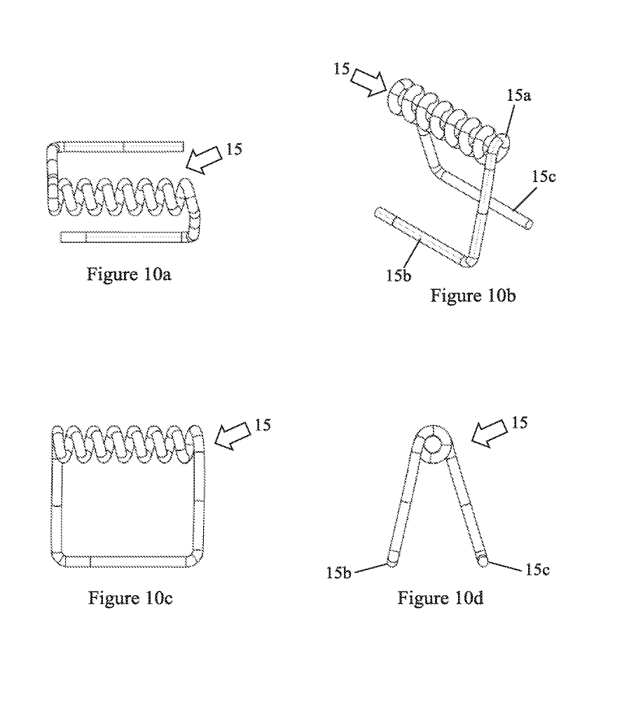

FIG. 10a-d: top, perspective side and end views of the spring used in the clip of the device shown in FIGS. 1-8.

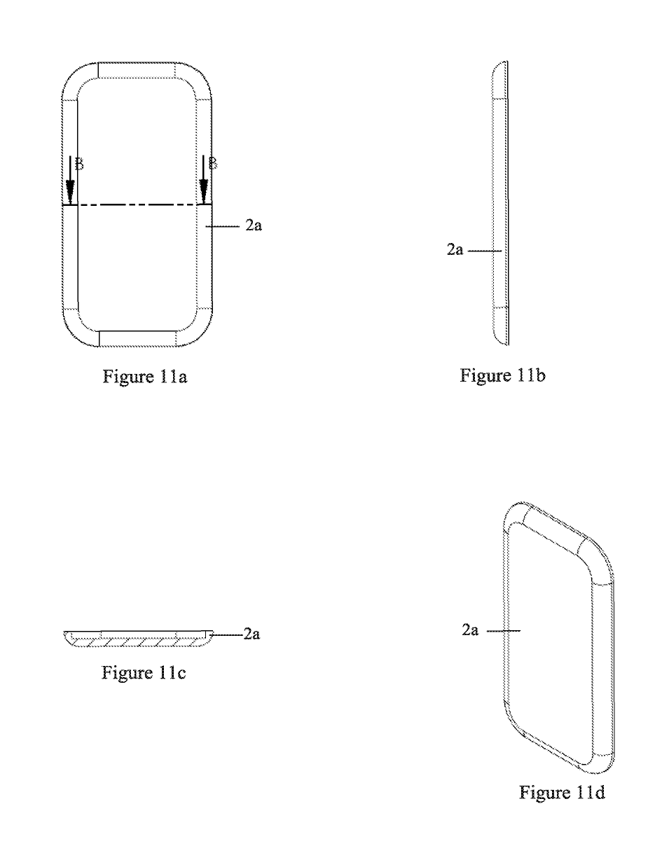

FIG. 11a-d: plan, side, cross-sectional and perspective views of the reservoir lid of the device shown in FIGS. 1-8.

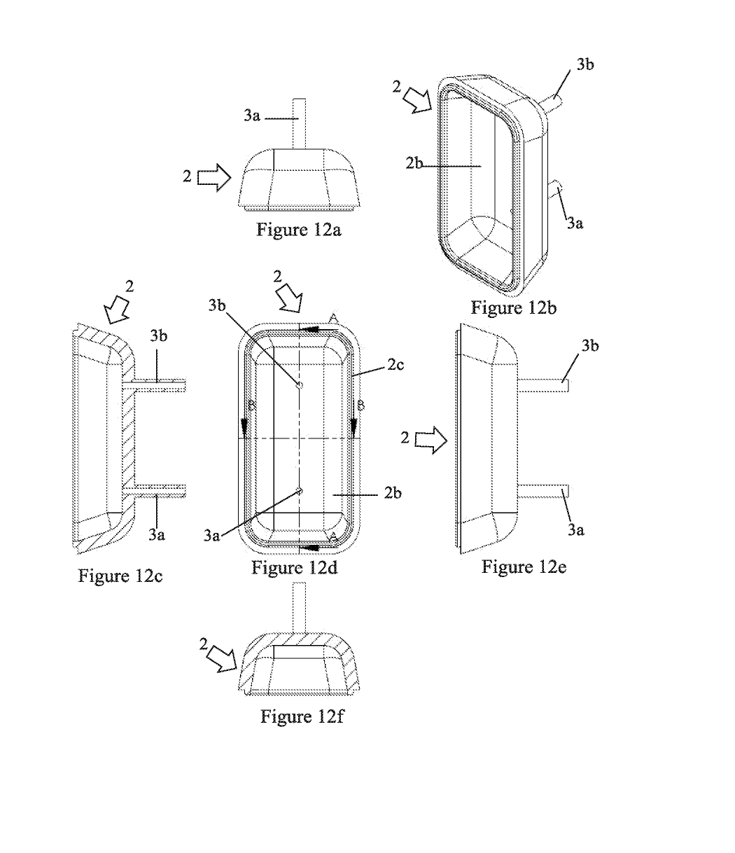

FIG. 12a-f: end, perspective, cross-sectional, plan and side views of the reservoir shown in the device of FIGS. 1-8.

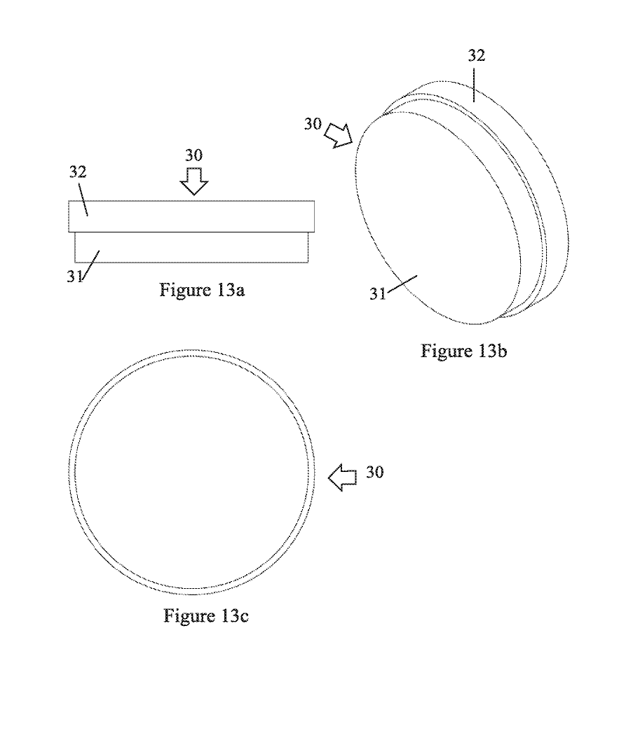

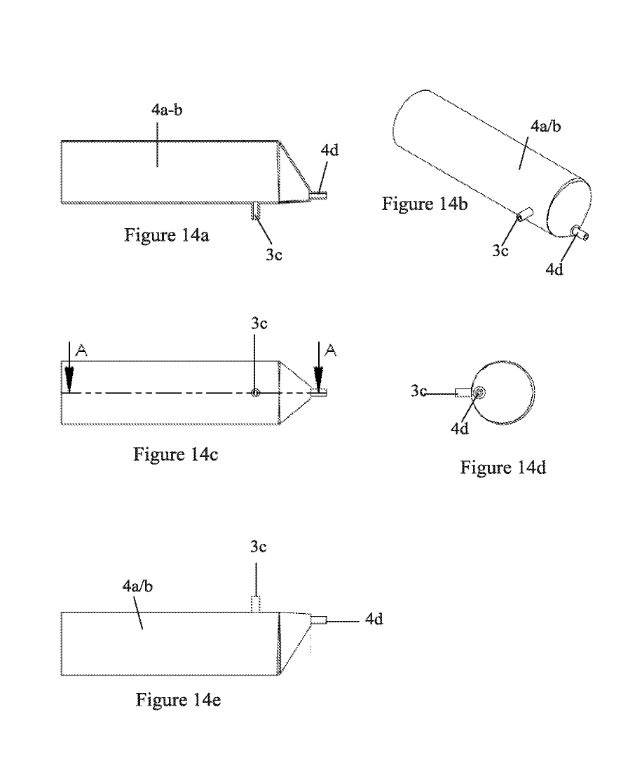

FIG. 13a-c: plan, perspective and end views of a canister cap FIG. 14a-e: cross-sectional, perspective, bottom, end and side views of a canister of the device shown in FIGS. 1-8.

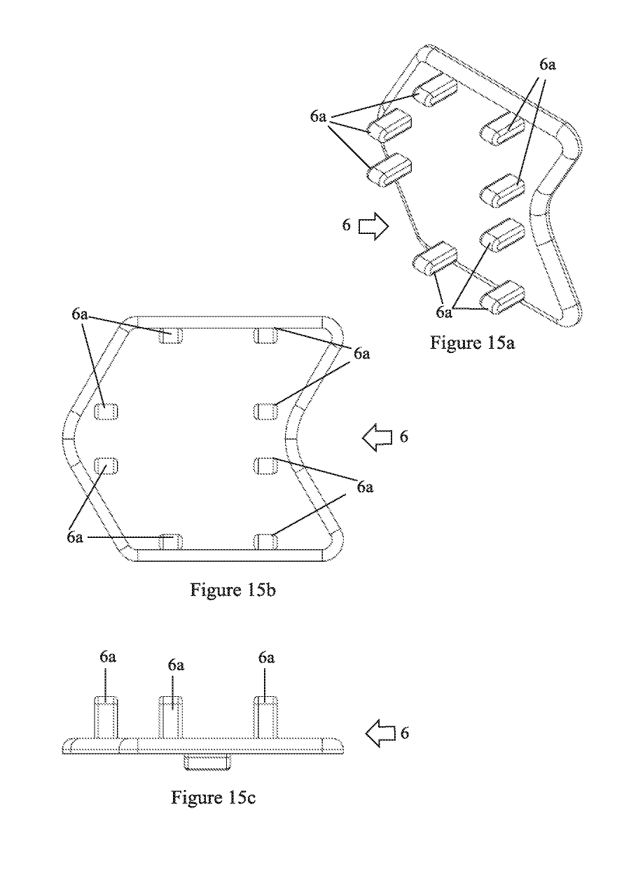

FIG. 15a-c: perspective, plan and side views of the base plate of the device shown in FIGS. 1-8.

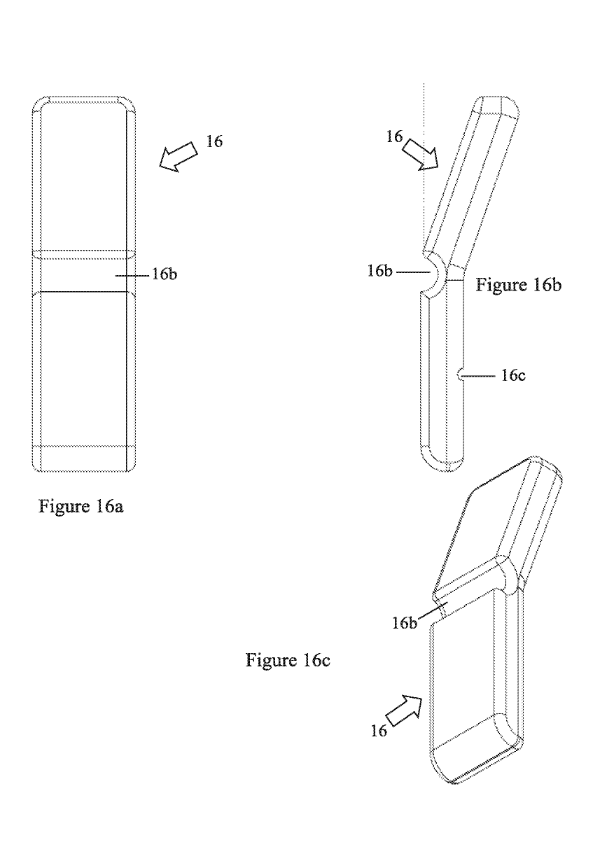

FIG. 16a-c: Top, side and perspective views of the actuator arm component of the clip shown in the device of FIGS. 1-8.

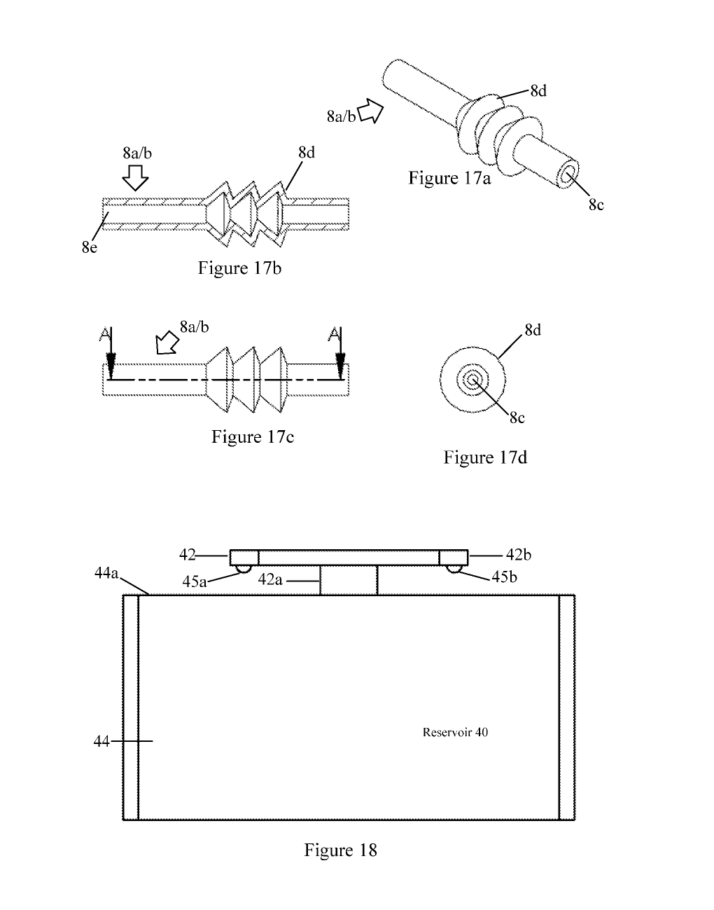

FIG. 17a-d: cross-sectional, perspective, side and end views of the flexible tubes of the device of FIGS. 1-8.

FIG. 18: A plan view of a reservoir component according to a further aspect of this invention.

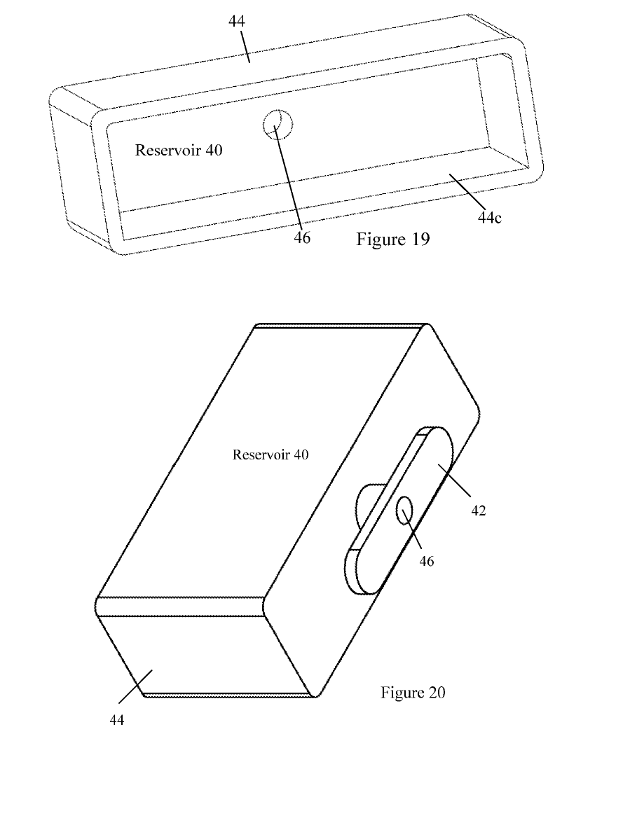

FIG. 19: A perspective view of the reservoir component of FIG. 18 showing the internal cavity for holding agent/fluid.

FIG. 20: A further perspective view of the reservoir component shown in FIGS. 18 and 19.

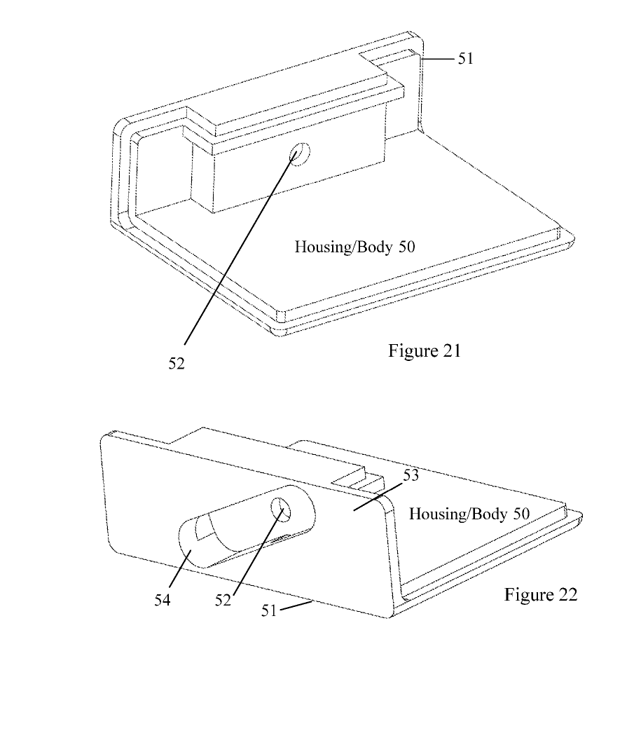

FIG. 21: A perspective view of a housing or body element adapted to be releasably connected to the reservoir component shown in FIGS. 18, 19 and 20.

FIG. 22: A further perspective view of the housing or body element shown in FIG. 21

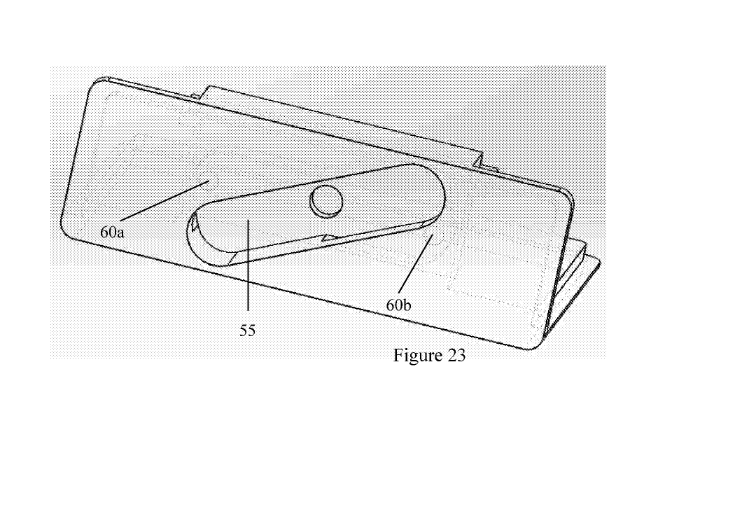

FIG. 23: A perspective “see-through” line drawing showing the lock aperture of the housing or body element.

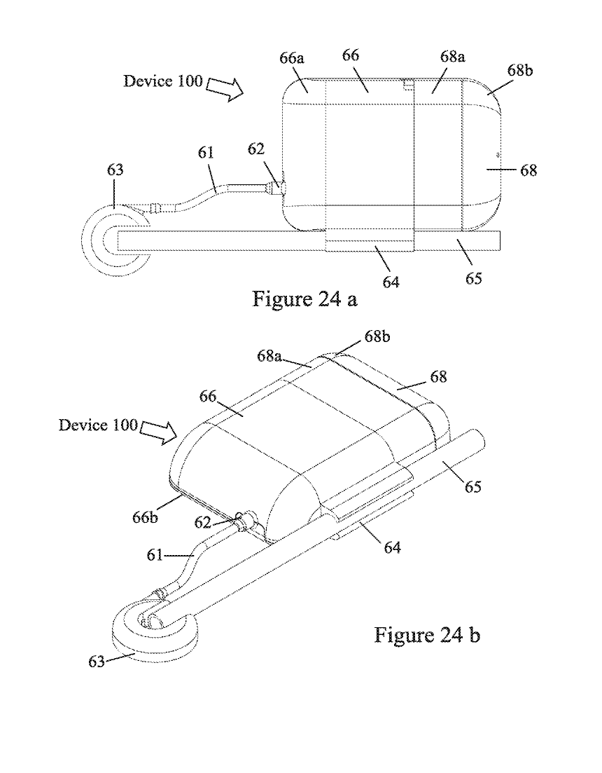

FIG. 24a: is a plan view of a device according to one embodiment of this invention.

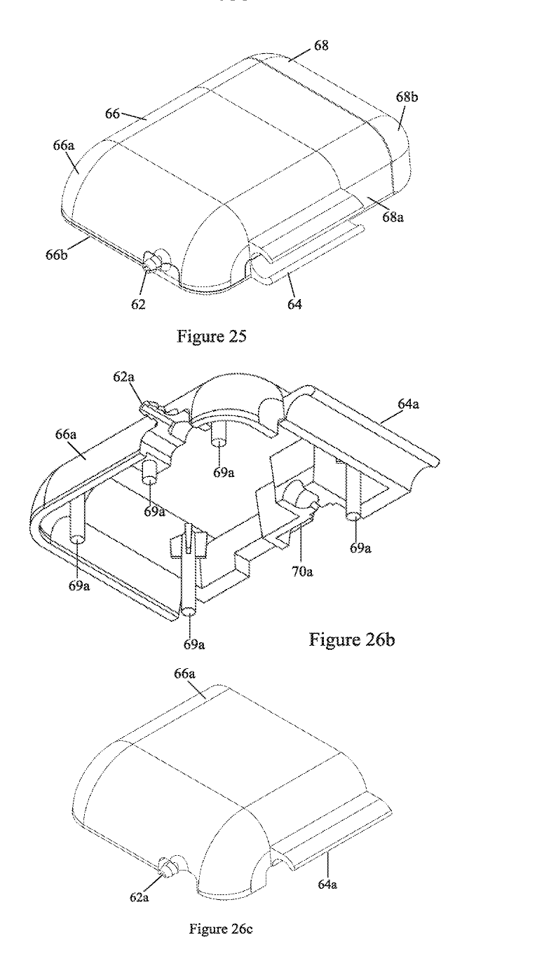

FIG. 24b: is a perspective view of the device shown in FIG. 24a FIG. 25: is a perspective view of the assembled body/housing and reservoir of the device of FIG. 24.

FIG. 26a-c: are perspective views of the bottom and top sides of the cover of the body/housing of the device shown in FIG. 24.

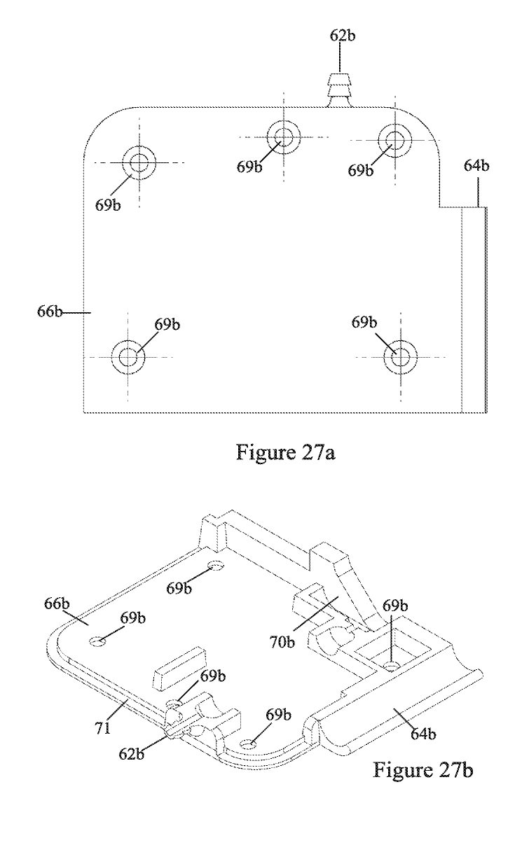

FIG. 27a-b: are bottom plan and perspective views of the base of the body/housing of the device shown in FIG. 24.

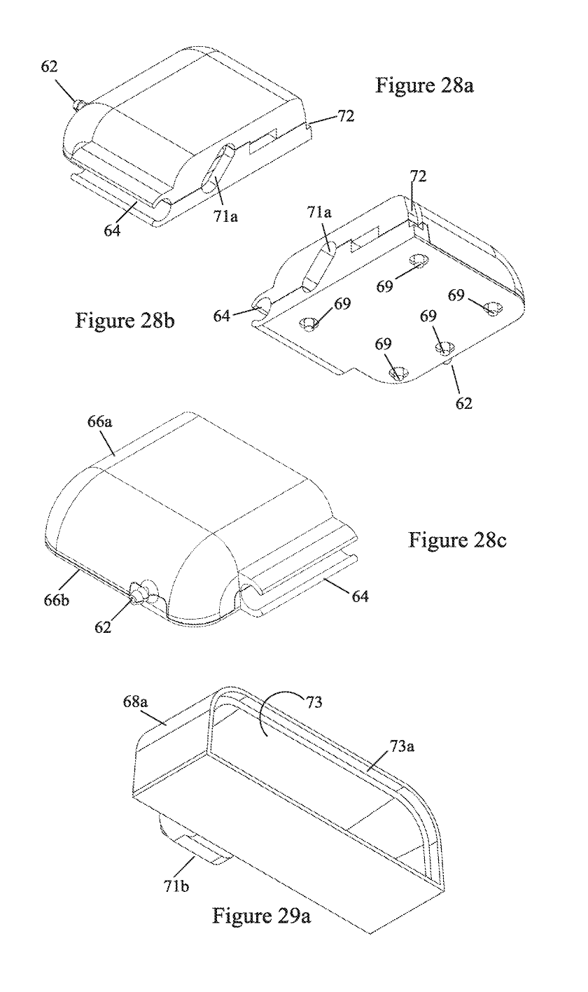

FIG. 28a-c: are perspective views of the assembled body/housing of the device shown in FIG. 24.

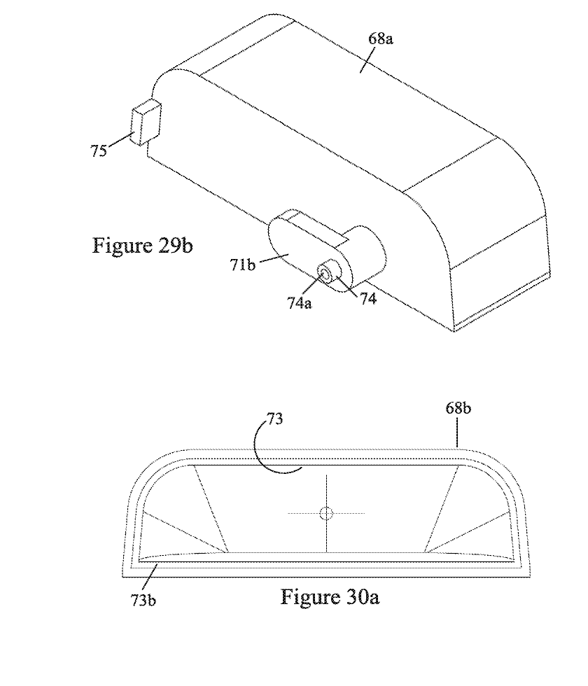

FIG. 29a-b: bottom and top perspective views of part of the reservoir of the device shown in FIG. 24.

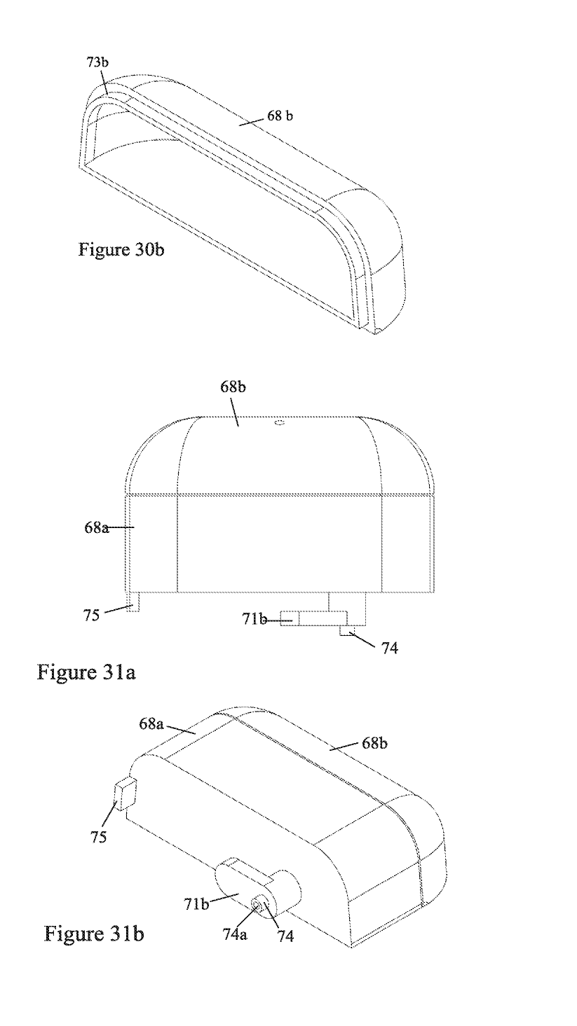

FIG. 30a-b: front view and side perspective of a second part of the reservoir of a device of FIG. 24.

FIG. 31a-b: top and perspective views of the reservoir component of the device shown in FIG. 24.

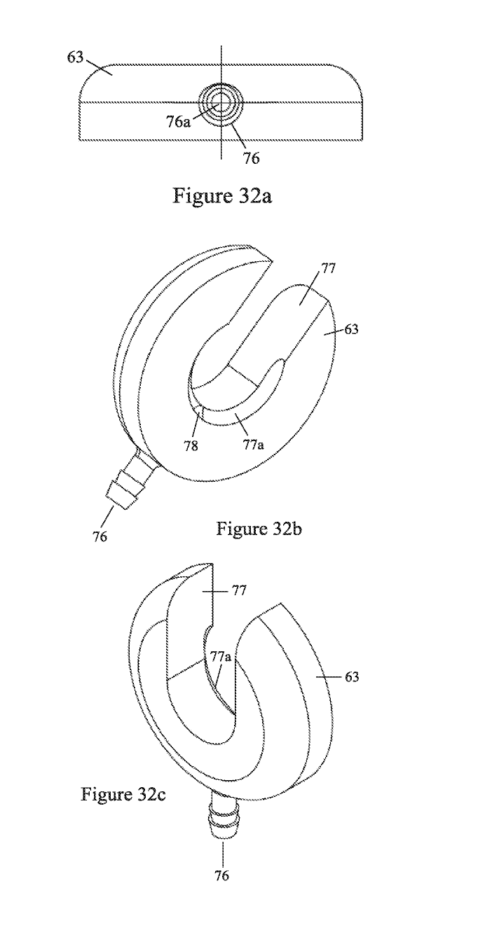

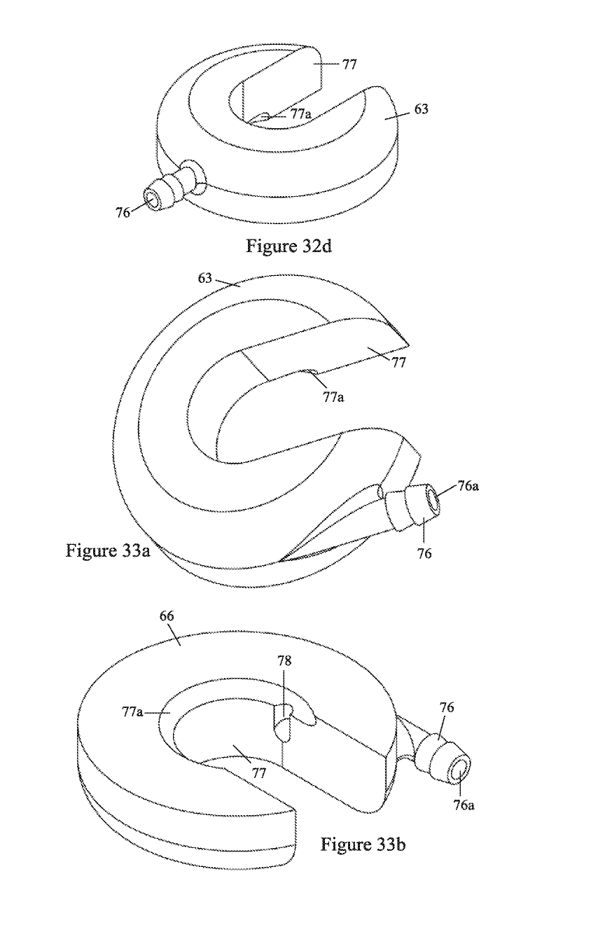

FIG. 32a: front plan view of the partial annulus nozzle of a device of this invention with the inlet port (68) centred.

FIG. 32b-d: perspective views of a partial annulus nozzle clip of a device of this invention.

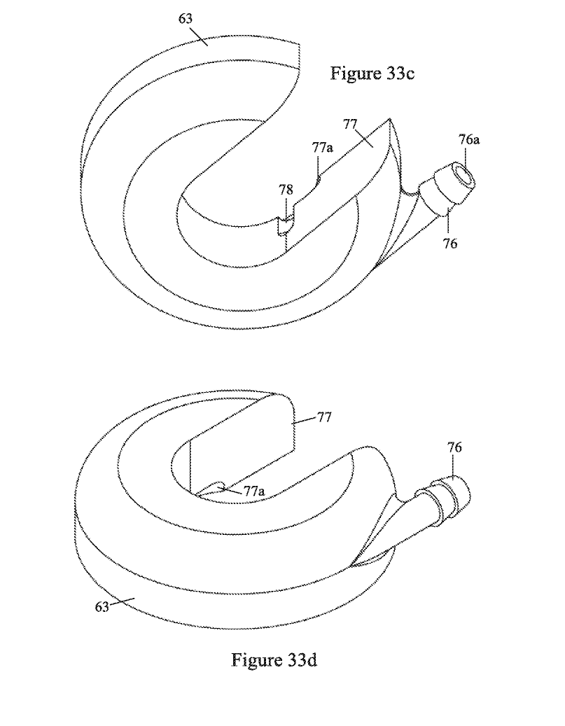

FIGS. 33a-d: perspective views of a partial annulus nozzle clip of the device shown in FIG. 24.

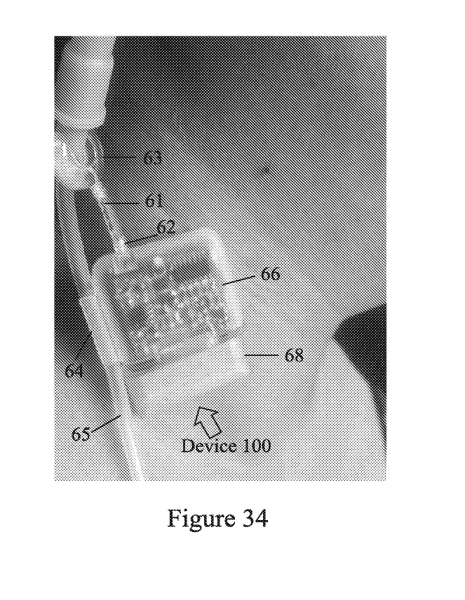

FIG. 34: picture of Device 100 in use.

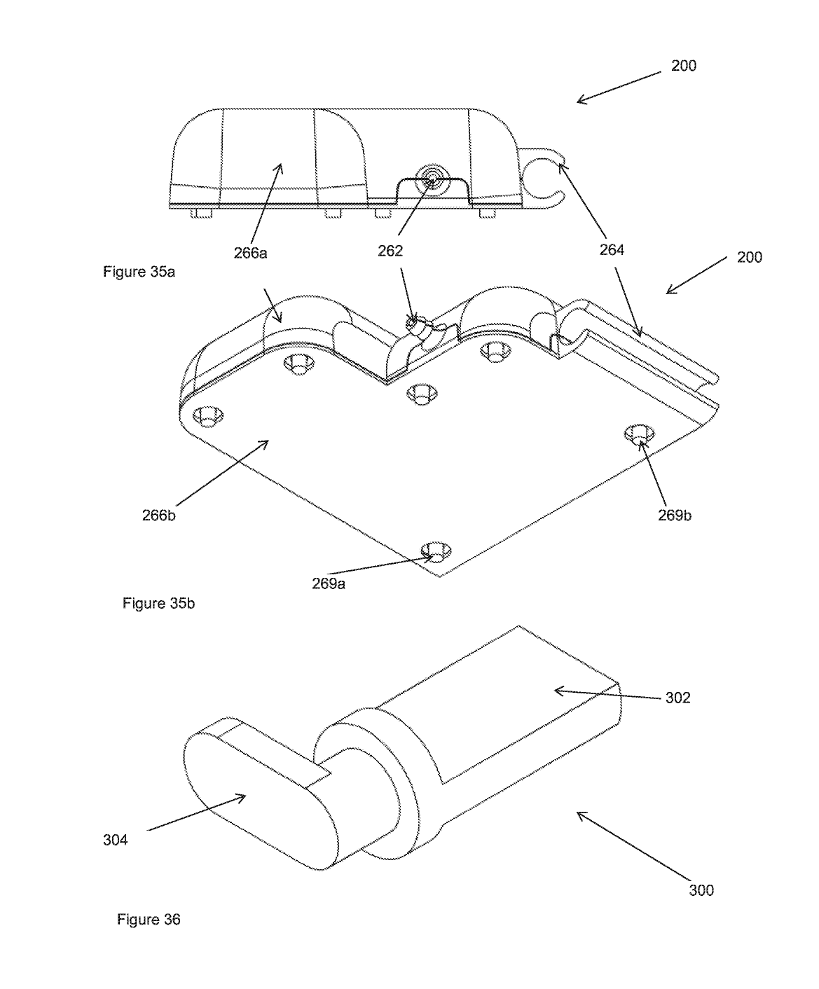

FIGS. 35a and b: Front plan and perspective views of a body section of a device according to an embodiment of this invention.

FIG. 36: a locking member for use with devices of this invention.

FIG. 1 is an exploded perspective view of an automated device 10 for applying, delivering, discharging or dispensing an agent to a wound. Device 10 comprises reservoir 2 connected by tubes 3a and 3b to canisters 4a and 4b. The reservoir has a releasable lid 2a and may be filled with an agent. Tubes 3a and 3b define conduits through which agent may flow or pass. As such, agent stored or held in the reservoir is fed to the canisters 4a and 4b via tubes 3a and 3b. Canisters 4a and 4b are mounted on a base plate 6 which comprises a plurality of lugs or prongs 6a which retain and hold (or grip) canisters 4a and 4b in position. Canisters 4a and 4b further comprise flexible tubes 8a and 8b (not shown here) through which agent is dispensed or discharged. The dispense or discharge of agent through flexible tubes 8a, is achieved by pistons 12a and 12b. Pistons 12a and 12b apply a pressure to agent fed into canisters 4a and 4b. This pressure forces agent through flexible tubes 8a and 8b.

Turning now to FIG. 2, the arrangement of the reservoir 2, 2a, canisters 4a and 4b, base plate and lugs 6, 6a and flexible tubes 8a and 8b can be seen.

Best shown in FIG. 3 is flexible tube 8b of device 10. The tube can be seen extending from the end of canister 4b.

FIG. 4 shows clip 14 located beneath base 6. Clip 14 contains a spring 15 which biases the clip to the closed position. In use, the actuator arm 16 of the clip is pressed to open the clip jaws 18. The clip 14 can then be used to attach device 10 to another article, for example, another piece of medical equipment.

FIG. 5 shows the side view of automated device 10. Best shown in this figure is reservoir 2 which is connected to canister 4a by tube 3a. Fluid contained within reservoir 2 can flow through tube 3a (under the action of gravity) into canister 4a, where upon it is ejected through flexible tube 8a. Canister 4a (and 4b not shown) is mounted on base plate 6 and retained in position by a plurality of griping lugs 6a. In this embodiment, the canisters 4a/4b may be released or separated from the base plate 6 and the lugs 6a thereof, by simple pulling action.

FIG. 6 shows the bottom of device 10. In this figure, clip 14 can be seen attached to base plate 6.

Turning now to FIG. 7, there is shown a front end view of device 10. In this figure, reservoir 2 can be seen with the lid 2a removed (shown above). As explained above, reservoir 2 is connected to canisters 4a and 4b via tubes 3a and 3b. These tubes (3a and 3b) form conduits through which fluid held in reservoir 2 may flow into canisters 4a and 4b. Base plate 6 is also shown upon which canisters 4 and 4b are mounted and (releasably) retained by lugs 6a. FIG. 7 also clearly shows clip 14 mounted on the bottom of base plate 6 which may be used to attach the device to other forms of equipment—such as, for example, IV drip lines and the like.

FIG. 8 is a cross-sectional view of one of the canisters 4a/4b. This view clearly shows that canisters 4a/b comprise a port 3c for engaging with or receiving tubes 3a and/or 3b. Port 3c defines an aperture opening into the interior chamber 4c of canister 4a/4b. Fluid passing from the reservoir 2 (not shown) though either of tubes 3a or 3b may enter the canister interior chamber (4c) via port 3c. Chamber 4c is formed an adapted to receive and retain fluid from reservoir 2. FIG. 8 also shows that canisters 4a/b further comprise a cap 30 and a piston 20 which is slidably mounted therein. Piston 20 may be driven or moved towards flexible tube 8a/b so as to apply pressure to fluid within chamber 4c. As piston 20 is moved to apply pressure to fluid within chamber 4c, the fluid if forced through flexible tube 8a/b. Once ejected from flexible tube 8a/b, the fluid can be directed to, for example a wound (for example a surgical puncture wound). As explained in more detail below (with reference to FIG. 15) direction of ejected fluid to a wound may be assisted by the orientation of flexible tubes 8a/b which may be angled to eject fluid in a particular direction.

FIG. 9 shows piston 20 in detail. Piston 20 comprises a cylindrical head 21 having a diameter substantially equal to the internal diameter of canisters 4a and 4b. Piston 20 may comprise one or more seal elements to form a seal between the outer surface of the cylindrical head 21 and the inner surface of chamber 4c of canisters 4a/4b. Piston 20 further comprises a shaft 22 which may be connected to, or engaged with a drive means. For example, shaft 22 may engage with a cam or other actuator element. FIG. 9c is an end view of piston 20 showing the head 21 and shaft 22 sections.

FIGS. 10a-d show the spring 15 of clip 14. Spring 15 may be formed of spring wire (perhaps of surgical grade) and comprises coiled spring section 15a and arms 15b and 15c which are connected to coiled spring section 15a and are biased to the open position thereby. Arms 15b and 15c engage with clip parts 16a and 16b.

FIGS. 11a-11d show the reservoir lid 2a in detail. The lid can be releasably attached to reservoir 2 and may form an air/water tight seal therewith. Lid 2a may comprise one or more seal elements such that when fitted to reservoir 2, the lid forms a water/air tight seal therewith.

Turning now to FIG. 12, there is shown various views of the reservoir 2. In FIGS. 12a and 12e which are end and front views respectively of reservoir 2, tubes 3a and 3b are shown. Through these tubes, fluid held within reservoir 2 may flow by gravity. FIG. 12b is a perspective view of the reservoir and shows internal chamber 2b which holds fluid. FIG. 12c is a cross-sectional view of the reservoir. Best shown here are the conduits defined by tubes 3a and 3b which open to the internal chamber of reservoir 2. In FIG. 12d, the openings defined by tubes 3a and 3b can be seen at the base of reservoir chamber 2b. Additionally, upper rim 2c of the reservoir is shown—the rim defines a lip which engages/mates with reservoir lid 2a. The rim and/or lip 2c of reservoir 2 may further comprise a seal element so that when in situ, lid 2a forms an air/water tight seal therewith.

FIG. 13, shows the canister cap 30 in detail. Canister cap 30 is placed as a bung or plug in the end of canister 4a or 4b, behind piston 20. Cap 30 is generally cylindrical in form and comprises a first cylindrical part 31 that is inserted into the chamber 4c of the canisters 4a and 4b. The diameter of part 31 is substantially identical to the internal diameter of chamber 4c. Part 31 may comprise one or more seal elements such that, in use, the cap forms an air/water tight seal with the canister 4a/4b.

FIGS. 14a-e best show the cylindrical canisters 4a and 4b. FIG. 14 a shows a cross-section through plane A-A of FIG. 14c and shows the internal chamber 4c of a canister 4a/b and port 3c which connects to one of tubes 3a or 3b. The canister further comprises nozzle 4d which is designed to engage with or connect to, the flexible tubes 8a and 8b. The internal conduit defined by nozzle 4d communicates with chamber 4c of the canister.

FIGS. 15a-c provide various views of base plate 6. As described above, base plate 6 comprises a plurality of lugs 6a which are positioned in such a way such that they grip and retain the canisters of device 10 in position. The lugs may comprise a coating to increase their grip on the canisters. For example, the lugs 6a may be coated with a layer of rubber or the like. The base plate 6 and/or lugs 6a may be formed of metal, plastic, wood, rubber, composite material (fibre glass, carbon fibre or the like) or any combination thereof.

FIG. 16 shows the detail of certain parts of the clip 14. In particular, the actuator arm 16 is shown. The actuator arm is bent near the middle and comprises an elongate body member 16a which defines a groove 16b for receiving the coiled spring 15a part of spring 15 and a further groove 16c for receiving one of the spring arms 15b or 15c.

Best shown in FIGS. 17a-d are the flexible tubes 8a and 8b. These tubes (8a and 8b) are generally elongate tubes which define central conduits through which fluid may pass. FIG. 17b (which is a cross-sectional view of a tube 8a or 8b through plane A-A of FIG. 17c) shows the conduit 8e defined by the tube. In these embodiments, the conduit defines a fluid exit aperture 8c which, in certain embodiments may be adapted to form a spray or jet of fluid. Also shown are the sections of tubes 8a and 8b which comprise corrugations 8d which permit flexing of the tube. Corrugations 8d permit flexing of tubes 8a or 8b while at the same time preserving the integrity and structure of the conduit 8e such that when flexed, fluid can still pass through tube 8a or 8b. The corrugations allow tubes 8a and 8b to be angled such that fluid ejected therefrom (through fluid exit 8c) may be directed to a wound. FIG. 17d is an end view showing the fluid exit 8c defined by one end of tube 8a or 8b and the corrugated portion 8d.

Turning now to FIGS. 18-23, there is shown an alternate embodiment of a reservoir component and the body/housing section of a device II according to this invention.

FIG. 18 is a plan view of reservoir component 40. The reservoir comprises body portion 44 which is formed and adapted to receive and hold a volume of agent or fluid. On its bottom surface 44a, the reservoir body 44 comprises projection 42 which itself comprise a shaft 42a protruding from the bottom surface 44a or the reservoir and a planar “key” section 42b. The “key” section 42b further comprises domed protrusions 45a and 45b.

FIG. 19 is a perspective view of reservoir 40 shown in FIG. 18. This view best shows the internal cavity 44c defined by reservoir body 44, which is adapted to hold and retain a quantity or volume of agent or fluid. Additionally, this figure shows aperture 46 defined by the bottom surface of internal cavity 44c. Aperture 46 opens into a bore defined by shaft 42a and key structure 42 (not shown). Fluid or agent retained within reservoir 40 may pass through the bore defined by shaft/key 42a/b. The bore may comprise a valve—perhaps a one-way valve.

FIG. 20 is a yet further perspective view of reservoir 40. Best shown here is aperture 46 which extends through the lower surface of the reservoir body 44, through shaft 42a and through key structure 42b.

FIG. 21 is a perspective view of a housing or body component 50 to which reservoir 40 may be connected. Housing/body 50 comprises an L-shaped structure having profiled wall 51. Profiled surface 52 defines aperture 52. The remaining features of this structure are best described with reference to FIG. 22 which shows profiled wall 51 to define a locking aperture 54. Locking aperture 54 defines an aperture on the outer surface 53 of profiled wall 51 which is adapted to receive key structure 42b. Aperture 52 opens out into locking aperture 54.

FIG. 23 shows the locking aperture in detail. In this Figure, the locking aperture can be seen to define a further cavity 55 which, like locking aperture 54, is adapted to receive key 42b. In use, key 42b is inserted into locking aperture 54 and pushed until it reaches the profiled wall. Key 42b is then turned such that key 42 is received by cavity 55. a surface of cavity 55 define domed recesses 60a and 60b—these recesses receive domed protrusions 45a and 45b to secure key 42b within cavity 55. Since Key 42b is fixed to reservoir 40, the reservoir becomes releasably connected (or locked) to body housing portion 50.

FIGS. 24a and b are perspective views of an automated device 100 for applying, delivering, discharging or dispensing an agent to a wound. As stated, in use, the device permits would visualisation and attendance without the need to move or displace the device. Moreover, in use, the device does not substantially occlude, obscure or cover the wound. Device 100 comprises a housing or body 66 (comprising upper and lower connected pats 66a and 66b) which is (releasably) connected to reservoir 68 formed of units 68a/68b, which reservoir (68) is capable of storing an agent, for example a fluid agent) to be delivered to a wound, ultimately through applicator or nozzle 63. Applicator nozzle 63 is in fluid communication with the housing 66 of the device through flexible tube 61, which is attached to fluid outlet 62 (located the housing/body component 66). The device is releasably attached to a catheter 65 via grip element 64 located on the side of the housing 66. Nozzle 63 is attached to catheter 65 such that it can deliver an agent (for example an antiseptic agent) to part of the catheter. In use, the agent held within reservoir 68 may be transmitted through the housing/body portion 66 of the device, through fluid outlet port 62 and flexible tube 61 to the applicator nozzle 63. Applicator nozzle 63 defines a fluid outlet aperture (not shown here) through which agent is dispensed, delivered or discharged. When applicator nozzle 63 is secured or attached to a catheter, agent may be dispensed, discharged or delivered from/by the device to the catheter. Moreover, when the applicator nozzle 63 is secured to a catheter at the point where the catheter enters, for example, the skin (i.e. at the wound), fluid dispensed, discharged or delivered from/by the device may also be applied to the wound. Again, where the agent is an antiseptic and/or anti-microbial agent, device 100 may be used to ensure that a catheter and/or wound (catheter entry site) remains clean and free of infection.

FIG. 25 shows the body/housing section 66 of the device attached to the reservoir section 68. In this Figure it can be seen that the body/housing part 66 comprises two sections 66a and 66b which are connected together. The reservoir section 68 also comprises two parts 68a and 68b which are connected together. Also shown is grip element 64 which protrudes from the side of the body portion 66 and is formed from connected parts of both sections 66a and 66b. In use, grip element 64 releasably may accommodate (by interference fit) a tubular member, for example a catheter. Also shown is fluid outlet port 62 which protrudes from a front surface of body section 66 and is formed from parts of both sections 66a and 66b of the body portion. Fluid outlet port 62 comprises a series of ridges or ribs such that a flexible tube (for example tube 61 in FIG. 24) may be releasably secured thereto. The ridges of the fluid outlet port 62 also ensure a fluid tight seal.

FIGS. 26a-c show perspective views of upper part 66a of the body section 66. FIGS. 26a and b show the inner surface of the body section 66a. The inner surface provides five cylindrical lugs 69a. The inner surface of part 66a further defines part of a locking aperture 70a. Also defined by part 66a is the top surface of fluid outlet port 62 which can be seen to define a channel through which fluid may flow. FIGS. 26a and b show the upper part of grip element 64a protruding from the side of the cover 66a.

FIG. 26c shows a top perspective view of body/housing part 66a. This figure clearly shows those parts (62a and 64a) of 66a which form part of the fluid outlet port 62 and grip element 64.

FIGS. 27a-b show bottom and perspective views of the other (or base) part 66b of body/housing section 66.

Base part 66b defines five apertures (69b) which are sized and dimensioned to securely accommodate the lugs (69a) projecting from the inner surface of part 66a. the apertures 69b defined by base part 66b are positioned such that when parts 66a and 66b are brought together, each of the lugs 69a become located within their corresponding apertures 69b. Parts 66a and 66b are held together by the resulting interference fit.

Best shown in FIG. 27b are those parts (62b, 64b and 70b) which form fluid outlet port 62, grip element 64 and locking aperture 70.

FIGS. 28a-b show perspective views of the assembled body section 66 formed from connected parts 66a and 66b. Best shown in FIGS. 28a and b is slot 71 which is adapted to receive part of the reservoir 68 (not shown). Also shown is step 72 which, in use, prevents over-rotation of the reservoir 68 when it is locked into place.

FIG. 28b shows the underside of housing 66 and each of the lugs 69 can be seen located within corresponding apertures 69b defined by part 66b.

FIG. 28c clearly shows the part of grip element 64 formed by parts 66a and 66b of the housing 66. Also shown is fluid outlet port 62 formed by parts 66a and 66b of the housing 66.

FIGS. 29a and b shows part 68a of the reservoir 68 in isolation. FIG. 29a shows that an inner surface 73 of reservoir part 68a defines a well which may contain fluid. The inner surface 73 further defines a ridge or step 73a which receives part of the reservoir lid 68. These features are also shown in FIG. 30a.

FIG. 29b clearly shows fluid outlet port 74 which protrudes from lock element 71b. Fluid outlet port 74 defines an aperture 74a which is in fluid communication with the well defined by inner surface 74 of part 67. Lock element 71b comprises a lateral lug which fits within slot 71a defined by housing 66 (not shown). In use, locking element 71b may be located within slot 71a such that it enters housing 66. Once locking element 71b has been located in slot 71a, it is necessary to align housing 66 with reservoir 68. This is achieved by rotating the reservoir. Rotating reservoir 68 has the effect of locking reservoir 68 to the housing 66 as the locking element 71b (now located within housing 66) becomes misaligned from slot 71a. To prevent over-rotation during the locking procedure, reservoir part 68a is provided with protruding lug 75 which meets step 72 as it is rotated thus preventing further (over) rotation.

FIG. 30b presents perspective view of part 68b. This part connects to part 68a to form a closed reservoir which may contain fluid. Reservoir 68 defines a lip 73b which permits an interference type connection with part 68a.

FIGS. 31a-b show plan and perspective views of the assembled reservoir comprising connected parts 68a and 68b.

FIGS. 32a-d show in detail a partial annulus applicator nozzle 63. FIG. 32a shows a centred fluid inlet port 76 which may be connected to flexible tube 61 (not shown). Fluid inlet port 76 is best shown in FIGS. 32b and 32c as comprising a profiled, ridged or ribbed surface which permits a secure and fluid tight seal with flexible tube 61. Fluid inlet port 76 defines an aperture 76a through which fluid from the flexible tube may pass. Fluid passing through aperture 76a may pass along bore 76b defined by an inner surface of fluid inlet port 76 and applicator 63. Fluid may exit the applicator via aperture 78 (shown in FIG. 32b) defined by an inner surface 77 of the applicator 63.

FIG. 32b shows a perspective view of applicator nozzle 63. In this Figure applicator 63 is shown to comprise a partial annulus structure. The partial annulus structure of applicator 63 defines a U shaped gap which is formed, dimensioned and adapted to receive, for example, medical equipment such as catheters, needles, tubes and the like.

The inner surface 77 of applicator 63 further defines a circumferential recessed surface (appearing as a bevel or groove) denoted numeral 77a which spans a distance either side of defined aperture 78. The recessed surface 77a facilitates the spread of fluid exiting aperture 78. Recessed surface 77a may define a channel (perhaps in co-operation with a medical device, for example a catheter); additionally or alternatively, recessed surface 77a may wick or draw fluid by capillary action and/or under gravity from aperture 78.

In use, applicator 63 may be placed on or around, or attached to a piece of medical equipment such as, for example, a catheter. For example applicator 63 may be placed on the skin such that a catheter or needle passes through the U-shaped gap defined by applicator 63. In this way, agent passed from the reservoir 68 of device 100 through aperture 78 defined by an inner surface 77 of applicator 63, is discharged, applied and/or dispensed on to part of the needle/catheter and/or a wound (for example a surgical puncture wound). The applicator 63 is formed and adapted to apply, discharge or dispense fluid around substantially all or at least a part of the external circumference of a needle and/or catheter. The combination of the design of the recessed (bevelled or grooved) surface 77a with the effect of gravity and/or any capillary and/or wicking forces, allows the coating of substantially the entire outer surface or any part thereof) of the medical equipment to which nozzle 63 is attached.

FIGS. 33a-c show another design of applicator nozzle 63a in which the fluid inlet port 76 is located on one side of the partial annulus of applicator 63a. As with applicator 63, applicator 63d defines a fluid conduit or bore defined by the body of the partial annulus and fluid inlet port 76. The inner surface 80 of the applicator 63a defines an aperture through which fluid (or agent) passing through the bore or conduit may exit. The inner surface 80 further defines a bevel or groove 80a which extends circumferentially either side defined aperture.

FIG. 34 shows a picture of Device 100 in use. The partial annulus 63 with a centred inlet port is placed on a tube 65 belonging to medical equipment (e.g. catheter). The fluid (for example medicated fluid) contained in reservoir 68 is pumped through the body/housing 66 and passes through conduit 62 to the flexible tube 61. The fluid travels from tube 61 to nozzle 63 and is dispensed onto the medical equipment 65. Recessed surface 77a (not shown) of nozzle 63 allows the fluid to be spread across part or the entire surface of tube 65 to provide disinfection at the wound site without hindering the wound visualisation. The complete device can be releasably attached to existing medical equipment above the wound site at different locations.

FIGS. 35a and b show front plan and perspective views of part 200 of another device according to an embodiment of this invention. Part 200 is a body part comprising upper and lower sections 266a and 266b which are connected together. Body part 200 has substantially the same features as those of equivalent parts shown in other figures, the principle difference being the “L-shape” of this body part 200 and the recessed nature of fluid outlet port 262 which is set back from the front edge. Specifically, the Figures show that body section 200 comprises two connected parts 266a and 266b which are L-shaped. An inner surface of upper part 266a (not shown) comprises six projecting lugs 269a which in FIG. 35b can be shown as received within corresponding apertures 269b defined by lower part 266b of the body section 200. As with other embodiments, body section 200 comprises grip element 264 which allows it to be releasably connected to medical/surgical devices/equipment.

FIG. 36 shows a perspective view of a sealing member 300 which can be inserted into slot 71a (or its equivalent feature in other embodiments) when no reservoir section is attached. This fills and/or plugs slot 71a and seals the body unit of the various devices of this invention. Sealing member 300 comprises lug 304 which can be inserted into slot 71a and handle 302 which may be used to turn the sealing member 300 so as to misalign lug 304 with slot 71a locking member 300 in place.

Those skilled in the art will recognise that the above-described embodiments are merely exemplary of the present invention and that various modifications and improvements may be made thereto without departing from the scope of the invention. Furthermore, the various embodiments described above include a number of different features. It will be recognised by those of skill in the art that many of these features offer advantages independently of the other features present in the embodiments and could be incorporated in other aspects of the invention. For example, the automated device of this invention may comprise a plurality of tubes like those shown as features 8a and 8b above. For example, the device may comprise any number between 1 and 100 tubes, each of which communicate with one or more of the canisters 4a and 4b. Each tube may define a fluid conduit and each tube may be flexible such that fluid ejected therefrom may be targeted to a specific site or sent (for example sprayed) in a particular direction. The tubes may be substantially straight (but flexible) and/or branched such that one tube connected to the canister branches into a plurality of fluid conduits.

1. An automated device for applying, delivering, discharging or dispensing an agent to a biological tissue, wound and/or medical/surgical equipment.

2. A device for automatically delivering an agent to a biological tissue, wound and/or medical/surgical equipment, said device comprising a reservoir for storing an agent to be delivered to a wound and/or medical/surgical equipment and an applicator for delivering, discharging and/or dispensing the agent to the wound and/or medical/surgical equipment.

3. The device of any preceding claim, wherein in use, the device does not occlude, obscure or cover the wound.

4. The device of claim 2, wherein the applicator fits around a cannula and/or catheter.

5. The device of any one of claims 2 to 4, wherein the applicator defines at least one agent outlet aperture.

6. The device of claim 5, wherein the applicator defines a channel extending from the fluid outlet aperture and through which agent discharged or dispensed from the applicator, may flow.

7. The device of claim 6, wherein agent flows through the channel as a result of capillary forces and/or a wicking action.

8. The device of any one of claim 6 or 7, wherein in use, the channel forms a capillary gap with a piece of medical/surgical equipment, cannula and/or catheter.

9. The device of any preceding claim, wherein the device is releasably connectable to a piece of medical/surgical equipment.

10. The device of any preceding claim, wherein the device is releasably connectable to an intravenous line, a catheter, a tube and/or a needle.

11. The device of any preceding claim, wherein the agent comprises an antimicrobial agent, an antibiotic, an antiseptic, cleaning agent, disinfectant and/or a drug.

12. The device of claim 1, wherein the device comprises a reservoir for storing said agent and means for automatically applying a volume of the fluid to the biological tissue wound and/or medical/surgical equipment.

13. The device of any preceding claim, wherein the wound is a surgical wound and/or puncture wound.

14. The device of claim 13, wherein the puncture wound is caused by insertion of a medical/surgical equipment.

15. The device of any one of claims any preceding claim, wherein the biological tissue is the skin, a mucosal tissue, an epithelial tissues or a surface of the gut.

16. The device of any one of claim 1-12 or 14, wherein the medical/surgical equipment is a needle, tube, cannula or catheter

17. The device of any preceding claim, wherein the device is automated.

18. The devices of any preceding claim, wherein the device may be programmed to deliver, discharge and/or dispense a predetermined quantity of agent at a predetermined time or times.

19. The device of any preceding claim, wherein the device comprises one or more environmental sensors such that the delivery, discharge and/or dispense of an agent to a wound or medical/surgical device is activated in response to one or more environmental stimuli.

20. The device of any preceding claim, wherein the device comprises a connector for gripping or attaching to other equipment and or a body part.

21. The device of claim 2, wherein the reservoir and applicator are connected by a flexible conduit.

22. The device of claim 2 or 21 wherein, in use, medicated fluid stored in the reservoir is communicated to the applicator, optionally through the flexible conduit.

23. The device of any one of claim 2 or 21-22, wherein the reservoir and/or applicator is releasably connectable to a piece of medical or surgical equipment.

24. The device of claim 23, wherein, in use, the reservoir is releasably connected to one part of a piece of medical/surgical equipment and the applicator to another part of the same piece of medical/surgical equipment.

25. The device of any one of claims 23, wherein the medical or surgical equipment is an intravenous line and/or catheter/cannula.

26. The device of claim 1 or 2, wherein the device is for applying, delivering, dispensing or discharging an agent to surfaces, contact surfaces, air and/or materials.

27. The device of any preceding claim, wherein the device comprises one or more device status and/or performance indicators.
